(19) United States
(12) Patent Application Publication
(10) Pub. No.: US 2026/0341471 A1
(43) Pub. Date: Oct. 29, 2026

(54) AUTOMATED NETWORK SELECTION FOR ULTRASOUND

(71) Applicant: KONINKLIJKE PHILIPS N.V., EINDHOVEN (NL)

(72) Inventors: Jochen Kruecker, Andover, MA (US); Naveen Balaraju, Dorchester, MA (US); Balasundar Iyyavu Raju, North Andover, MA (US)

(21) Appl. No.: 19/103,864

(22) PCT Filed: Aug. 14, 2023
(86) PCT No.: PCT/EP2023/072352

§ 371 Date: Feb. 14, 2025

Related U.S. Application Data
(60) Provisional application No. 63/400,130, filed on Aug. 23, 2022.

(51) Int. Cl. G16H 30/20; A61B 8/00; G06N 3/045; G06N 3/08

(52) U.S. Cl. CPC G16H 30/20; A61B 8/5223; G06N 3/045; G06N 3/08

(57) ABSTRACT

Ultrasound image devices, systems and methods are provided. A system is configured to process newly acquired images of a subject by implementing a neural network trained specifically to process images of that subject. The image acquisition settings applied to image the subject, such as gain and/or scan depth, are used to determine which broader subject class corresponds to the subject. Determination of the appropriate class triggers implementation of a neural network associated exclusively with that class, such that images acquired from the subject are input to the neural network for processing. A plurality of neural networks can be used to perform a given function using acquired ultrasound images, and because each neural network may be trained using only a subset of images corresponding to a unique subject class, each network may be smaller, faster and more efficient than larger networks required to process images from a wider variety of subjects.

### TECHNICAL FIELD

The present disclosure pertains to ultrasound systems and methods for automated neural network selection based on classification of a subject to be imaged. Implementations involve determining a subject class corresponding to an imaged subject and inputting images obtained therefrom to a class-specific neural network for processing.

### BACKGROUND

Artificial intelligence algorithms are being developed to support ultrasound systems with automated procedure guidance, workflow automation, and image interpretation assistance. Convolutional neural networks, in particular, may be relied upon to perform complex tasks in substantially real time using ultrasound images obtained during execution of a scan protocol. A great variety of images are typically required to train such networks due to the diversity of images that the network will be expected to process correctly during the application phase. This diversity stems largely from user-dependent, manual image acquisition, as well as the large variety of anatomical and physiological characteristics found in different subjects, such as medical patients. Subject diversity further increases the variability of acquired images, which requires increasingly large training data sets and complex neural networks. It is expensive and challenging to develop and train networks that are adequately robust to provide accurate results for a wide variety of possible input images, let alone at the speed required to execute in real time on pre-existing ultrasound system hardware having limited storage space. These impediments are especially pronounced for handheld ultrasound systems running on mobile devices, which are increasingly common for point-of-care applications.

Improved technologies are therefore needed to quickly process large numbers of diverse ultrasound images using neural networks.

### SUMMARY

The present disclosure describes ultrasound systems configured to classify imaging subjects based at least in part on the ultrasound system settings used to acquire images therefrom. The disclosed systems may input newly acquired images into class-specific neural networks each trained using a distinct set of images corresponding only to its assigned class. Each class may be defined by a unique set of criteria such that images assigned to a certain class are more similar to each other than to images assigned to a different class. Each neural network can be selected from a plurality of neural networks, with each neural network trained to process images from a different class. By sorting like images into separate classes and assigning only one class to one neural network, each neural network may be trained using a smaller set of images relative to the number of images required to train a neural network tasked with processing a larger number of images having a greater variety of characteristics. The use of smaller, more compact networks each dedicated to a single subject class may increase processing speed and reduce computational load with little to no user intervention, thereby enabling automated, real-time image processing with reduced data storage.

In accordance with at least one example disclosed herein, an ultrasound imaging system may include an ultrasound transducer configured to acquire echo signals responsive to ultrasound pulses transmitted toward a subject and one or more processors in communication with the ultrasound transducer. The processors may be configured to determine a subject class corresponding to the subject based on examination parameters comprising one or more image acquisition settings used to generate at least one image frame from the acquired echo signals. The processors may also be configured to select a neural network associated with the subject class from a plurality of neural networks each associated with a different subject class, and input the at least one image frame to the neural network, the neural network configured to generate an output.

In some examples, the system also includes a user interface configured to receive indications of the examination parameters. In some examples, the system also includes a controller coupled with the ultrasound transducer, the controller configured to implement the one or more image acquisition settings in the ultrasound transducer. In some examples, the examination parameters further comprise one or more characteristics of the subject. In some examples, the one or more characteristics of the subject comprise one or more of a body mass index (BMI), a weight, a developmental stage, an age, a height, or a health condition. In some examples, the examination parameters further comprise an examination type comprising one or more of: a lung ultrasound, a cardiac ultrasound, an abdominal ultrasound, an obstetric ultrasound, a pelvic ultrasound, a transabdominal ultrasound, a transvaginal ultrasound, a transrectal ultrasound, a vascular ultrasound, a liver ultrasound, a renal ultrasound, a transcranial ultrasound, or a thyroid ultrasound. In some examples, the one or more image acquisition settings comprise a gain, a scan depth, a probe type, a frequency, or a scan pattern.

In some examples, the user interface further comprises a display configured to display the output. In some examples, the output comprises one or more of an ultrasound image, an identification of a natural or artificial feature visible within the ultrasound image, or a user instruction for performing an ultrasound exam. In some examples, the one or more processors are configured to determine the subject class by applying one or more thresholds to the examination parameters. In some examples, the one or more thresholds comprise a range of depth settings included in the image acquisition settings. In some examples, each of the plurality of neural networks is operatively associated with a training algorithm configured to receive an array of training inputs and known outputs, wherein the training inputs comprise ultrasound image frames obtained from a distinct subject class.

In accordance with at least one example disclosed herein, a method of ultrasound imaging involves acquiring echo signals responsive to ultrasound pulses transmitted into a target region of a subject by a transducer operatively coupled to an ultrasound system. The method also involves generating at least one image frame from the echo signals and determining a subject class based on examination parameters comprising one or more image acquisition settings used to generate the at least one image frame from the acquired echo signals. The method further involves selecting a neural network associated with the subject class from a plurality of neural networks each associated with a different subject class, inputting the at least one image frame to the neural network configured to generate an output, and causing the output to be displayed on a user interface.

In some examples of the method, the examination parameters further comprise one or more characteristics of the subject. In some examples of the method, the one or more characteristics of the subject comprise one or more of a body mass index (BMI), a weight, a developmental stage, an age, a height, or a health condition. In some examples of the method, the image acquisition settings comprise a gain, a scan depth, a probe type, a frequency, or a scan pattern. In some examples of the method, determining the subject class comprises applying one or more thresholds to the examination parameters. In some examples of the method, the one or more thresholds comprise a range of depth settings included in the image acquisition settings. In some examples, the method further involves training each of the plurality of neural networks using an array of training inputs and known outputs, the training inputs comprising ultrasound image frames obtained from a distinct subject class.

Any of the methods described herein, or steps thereof, may be embodied in a non-transitory computer-readable medium comprising executable instructions, which when executed may cause one or more hardware processors to perform the method or steps embodied herein.

### BRIEF DESCRIPTION OF THE DRAWINGS

FIG. 1 is a block diagram illustrating an example of an ultrasound system configured for automated neural network selection in accordance with embodiments of the present disclosure.

FIG. 2 is a block diagram illustrating an example processor implemented in accordance with embodiments of the present disclosure.

FIG. 3 is a block diagram illustrating additional components of an ultrasound system configured for automated neural network selection in accordance with embodiments of the present disclosure.

FIG. 4 is a block diagram illustrating an example of an automated neural network selection process and the associated ultrasound system components used to implement the same in accordance with embodiments of the present disclosure.

FIG. 5 is a flowchart of a method of automated neural network selection implemented in accordance with embodiments of the present disclosure.

### DETAILED DESCRIPTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems, or devices. Accordingly, embodiments may take the form of a hardware implementation, an entirely software implementation, or an implementation combining software and hardware aspects. The following detailed description is, therefore, not to be interpreted in a limiting sense.

Reference in the specification to “one embodiment” or “an embodiment” means that a particular feature, structure, or characteristic described in connection with the embodiment is included in at least one embodiment of the invention. The appearance of the phrase “in one embodiment” in various places in the specification are not necessarily all referring to the same embodiment.

As used herein, the term “plurality” may refer to two or more items, components, or characteristics. For example, a plurality of neural networks can include two, three, four, five, six, or more neural networks.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. These quantities may take the form of electrical, magnetic, or optical signals capable of being stored, transferred, combined, compared and otherwise manipulated. It is convenient at times, primarily for reasons of common usage, to refer to these signals as bits, values, elements, symbols, characters, terms, numbers or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing,” “computing,” “calculating,” “determining,” “displaying” or the like, refer to the action and processes of a computer system, or similar electronic computing device, that manipulates and transforms data represented as physical electronic quantities within the computer system memories or registers or other such information storage, transmission, or display devices.

As used herein, “subject” may refer to a patient receiving outpatient or inpatient medical care, which may involve medical treatment or examination, such that a “subject class” may comprise a patient class in some examples. Notably, however, the term “subject” is not limited to medical patients; it may also refer to a human or animal being imaged by an imaging system for a variety of reasons in a variety of settings.

An ultrasound system according to the present disclosure may implement one or more neural networks, for example at least one convolutional neural network (CNN), deep neural network (DNN), or the like, which may be uniquely synced with data acquisition hardware and a user interface. Individual embodiments may utilize a group of two or more neural networks trained to perform the same or similar function, but using separate classes of ultrasound images as input. Systems not configured in the manner disclosed herein may require the creation and implementation of neural networks configured to generate the same output pursuant to a particular exam, but using one or more additional processing steps necessitated by variation between different subject populations and images acquired by different ultrasound operators. The function(s) performed by a given group of neural networks may vary, as embodiments of the disclosed systems may incorporate or be compatible with a variety of neural networks trained to perform an assortment of functions. For example, the neural networks can be trained to recognize certain features present within an ultrasound image, such as anatomical features, ultrasound artifacts (e.g., A- or B-lines), medical devices, etc. In addition or alternatively, the neural networks may be trained to recognize certain features present within obtained ultrasound images and in response, generate instructions for adjusting an ultrasound probe position and/or image acquisition setting to obtain images of improved clarity, images taken along a specific scan plane, or images required for a particular scan protocol.

The neural networks utilized according to the present disclosure may be hardware-(e.g., neurons are represented by physical components) or software-based (e.g., neurons and pathways implemented in a software application), and can use a variety of topologies and learning algorithms for training to produce a desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a variety of functions and generate a corresponding output for additional processing and/or display. The ultrasound system may include a display or graphics processor, which is operable to arrange the ultrasound image and/or additional graphical information, which may include a worklist of features to be imaged and/or measured, annotations, tissue information, patient information, indicators, and other graphical components, in a display window for display on a user interface of the ultrasound system. In some embodiments, the ultrasound images and associated information may be provided to a storage and/or memory device, such as a picture archiving and communication system (PACS) for reporting purposes, developmental progress tracking, or future machine training (e.g., to continue to enhance the performance of each neural network). In some examples, ultrasound images obtained during a scan may be selectively or automatically transmitted, e.g., over a communications network, to a specialist trained to interpret the information embodied in the images, e.g., an obstetrician-gynecologist, an ultrasound specialist, a physician, or other clinician, thereby allowing a user to perform the ultrasound scans in various locations.

An ultrasound system in accordance with principles of the present disclosure may include or be operatively coupled to an ultrasound transducer configured to transmit ultrasound pulses toward a medium, e.g., a human body or specific portions thereof, and generate echo signals responsive to the ultrasound pulses. The ultrasound system may include a beamformer configured to perform transmit and/or receive beamforming, and a display configured to display, in some examples, ultrasound images and associated output generated by the ultrasound imaging system.

Certain aspects of the present invention include process steps and instructions that could be embodied in software, firmware, or hardware, and when embodied in software, could be downloaded to reside on and be operated from different platforms used by a variety of operating systems. Embodiments can comprise one or more applications available over the Internet, e.g., software as a service (SaaS), accessible using a variety of computer devices, e.g., smartphones, tablets, desktop computers, etc.

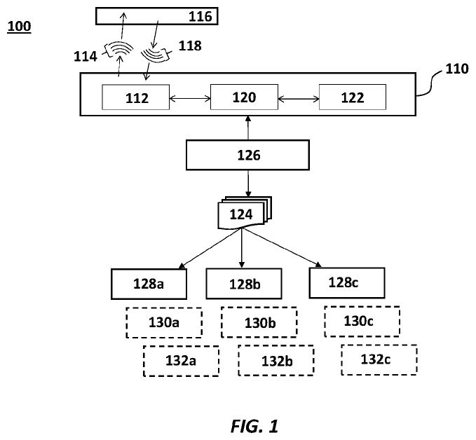

FIG. 1 shows an example ultrasound system 100 implemented according to principles of the present disclosure. The ultrasound system 100 may include an ultrasound data acquisition unit 110 comprising an ultrasound probe that includes an ultrasound sensor array 112 configured to transmit ultrasound pulses 114 into a region of a subject 116, e.g., chest region of a human, and receive ultrasound echoes 118 responsive to the transmitted pulses. As further shown, the ultrasound data acquisition unit 110 can include a beamformer 120 and a signal processor 122, which can be configured to generate a stream of discrete ultrasound image frames 124 from the ultrasound echoes 118 received at the array 112. The system 100 can also include a data processor 126, e.g., a computational module or circuity, configured to determine a subject class based on the ultrasound system settings applied to acquire the image frames 124 from the subject 116 and optionally one or more characteristics of the subject. Based on the determined subject class, the data processor 126 (alone, or in combination with one or more additional processors) can select and input the newly acquired image frames 124 into one of a plurality of neural networks 128a, 128b, 128c trained specifically to process images from that particular subject class. The neural networks 128a, 128b, 128c may be configured to perform a variety of functions using the input image frames 124, non-limiting examples of which can involve identifying natural or artificial features visible within the image frames, generating user instructions for acquiring additional image frames, etc. Because each neural network 128a, 128b, 128c may be trained to process image frames exclusively from one subject class, the network may be more compact and faster than a larger network trained to process a broader selection of image frames derived from a variety of different subject classes. Automatic determination of a subject class and selection of an appropriate neural network in view of the same may eliminate or at least reduce the need for user intervention during image processing, which would likely require extensive validation studies and regulatory filings.

As noted above, all of the neural networks 128a, 128b, 128c may be trained to perform the same function, such that the image frames 124 are processed in the same or similar manner regardless of network. Embodiments of the systems disclosed herein may be configured to implement a variety of different neural network groups, for example such that a first group of neural networks 128a, 128b, 128c is configured to perform one function, e.g., identify a certain anatomical feature within acquired image frames, a second group of neural networks 130a, 130b, 130c is configured to perform a different function, e.g., identify certain ultrasound artifact(s) from acquired image frames, a third group of neural networks 132a, 132b, 132c is configured to perform yet another distinct function, e.g., generate an alert based on identified anatomical features or artifacts, and so on. In some embodiments, automatic selection and implementation of one group of similarly trained neural networks from a larger set of neural networks may be initiated upon receipt of user input indicative of exam type. For example, selection of a lung imaging protocol may cause newly acquired image frames to be input to one group of neural networks trained to identify a pleural line, whereas selection of a fetal imaging protocol may cause newly acquired image frames to be input to a different group of neural networks trained to identify various anatomical features of a developing fetus. Additional, non-limiting examples of exam types may include cardiac ultrasound, abdominal ultrasound, obstetric ultrasound, pelvic ultrasound, transabdominal ultrasound, transvaginal ultrasound, transrectal ultrasound, vascular ultrasound, liver ultrasound, renal ultrasound, transcranial ultrasound, and/or thyroid ultrasound.

In operation, a subject class may be determined by the data processor 126 in view of the ultrasound imaging settings applied to image a particular subject 116. The settings may include depth, gain, focal zone, and/or frequency, among others. Relevant settings may also include probe type, e.g., linear, sector, or curvilinear, which vary in overall image geometry and beamforming, leading to differences in contrast and resolution across the depth of the resulting images. Subject characteristics or demographics may also be utilized to determine the subject class, non-limiting examples of which may include body mass index (BMI), weight, age, height, developmental stage, and/or one or more health conditions, among other factors. One or more of the subject characteristics can be input by a user or obtained from an electronic medical record, for example if the ultrasound system being used is connected to a hospital IT system. Collectively, the factors utilized to image the subject may correspond to a particular subject class associated with a neural network pre-trained using image frames obtained from a sample of images obtained from numerous subjects belonging to that subject class. The selected neural network is thus uniquely suited to process a certain subset of images.

Subject class determination may be performed in some embodiments by thresholding individual ultrasound settings or combinations of settings. For example, for a lung ultrasound exam, three neural networks may be trained: one for infant subjects, one for pediatric subjects, and one for adult subjects. Due primarily to the average differences in physical size between these subjects, the depth settings used to image them will likely be different. The different depth settings can be used to organize the imaged subjects by class, which can be used to input images acquired therefrom to particular class-assigned neural networks. Sorting and funneling images in this manner may be more efficient, fast, and accurate than processing all images from two or more classes using a single neural network.

The actual thresholds utilized to demarcate subject classes may vary according to exam type. By way of illustration, only, a depth setting of less than 5 cm used for a lung ultrasound exam may assign the subsequently acquired images to an infant class, a depth setting between 5 cm and 10 cm may assign the images to a pediatric subject class, and a depth setting of greater than 10 cm may assign the images to the adult subject class. The same or similar threshold limits can be used for acquiring additional image frames during the same exam, such that newly acquired images are assigned to a neural network in consistent fashion. Advantageously, the system may be configured to redirect newly acquired images obtained from the same subject into different neural networks “on the fly” upon modification of the ultrasound imaging settings. This real-time or substantially real-time adjustment is made possible at least in part by the increased processing speed enabled by utilizing a plurality of smaller, more efficient neural networks trained on specific image sets.

In a similar fashion, body type or BMI can be used to classify subjects into discrete classes. Images acquired from high-BMI subjects are usually marked by more noise and relatively low quality because of attenuation in subcutaneous fat layers. This attenuation often alters the appearance of the subject's underlying anatomy to the extent that separate neural networks may be needed to process images obtained from low-, normal-, and high-BMI subjects. Increased gain settings can be applied to compensate for higher attenuation typical of high-BMI subjects. The gain setting, then, can be used to distinguish between subject classes defined at least in part by body type, which may be further refined by additional ultrasound settings and/or subject characteristics.

If the final output or overall performance of one or more neural networks is determined to be suboptimal, the front-end thresholding used to determine subject class can be modified such that the image frames received by the underperforming neural network(s) may change. For example, thresholds applied to determine subject classes can be adjusted over time to improve the final system outputs by reviewing the accuracy and quality of ultrasound images and associated output(s) obtained from subjects of known class. One or more data processors and/or neural networks depicted in FIG. 1 can be configured to adjust a variety of thresholding limits in response to final output assessment, thereby creating an automatic feedback loop that iteratively modifies the thresholding limits in a manner that enhances the accuracy, sensitivity, and/or quality of the final outputs. Like the subject class determinations and neural network selections, iterative refinement of the thresholding limits can be performed automatically by one or more system components with little to no user intervention. In some examples, image statistics such as mean brightness and signal-to-noise ratio can be used in connection with gain setting to determine and refine identified body type, for example, as higher-BMI body types tend to correspond to darker and noisier images.

A variety of ultrasound systems, e.g., Philips LUMIFY, can be utilized and/or configured to implement the disclosed methods in real time or substantially real time. Mobile, handheld, or otherwise point-of-care ultrasound systems may be used to perform the disclosed methods, and may exhibit significant performance improvements due to the use of smaller, more specifically trained neural networks each requiring less storage space relative to preexisting neural networks employed for similar ultrasound image processing. In some examples, image processing and result output can be implemented on a system separate from the components utilized specifically for ultrasound image acquisition, including for example a cloud-based system, to which the ultrasound images and ultrasound settings are transferred wirelessly or via cable connection. In some embodiments, one or more of the acquisition components, e.g., ultrasound data acquisition unit 110, may be uniquely coupled to the components used for image processing.

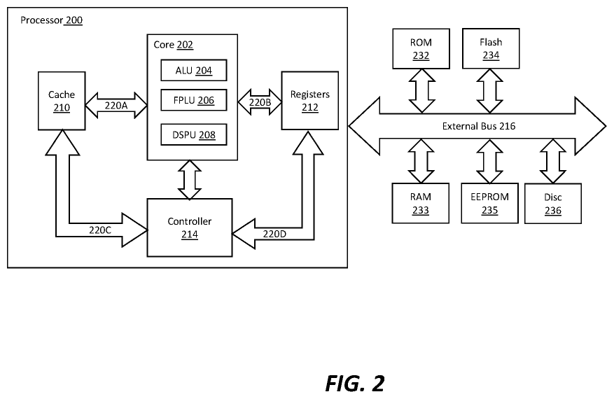

FIG. 2 is a simplified block diagram illustrating an example processor 200 according to principles of the present disclosure. One or more processors utilized to implement the disclosed methods, such as processor 126 of system 100, may be configured the same as or similarly to processor 200. Processor 200 may be used to implement one or more processes described herein, such as the automatic determination of a subject class (before and after initiation of an ultrasound exam), the automatic selection and/or implementation of a neural network, and/or the automatic adjustment of subject class attribute thresholds.

Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 200 may include one or more cores 202. The core 202 may include one or more arithmetic logic units (ALU) 204. In some examples, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some examples the registers 212 may be implemented using static memory. The register may provide data, instructions and addresses to the core 202.

In some examples, processor 200 may include one or more levels of cache memory 210 communicatively coupled to the core 202. The cache memory 210 may provide computer-readable instructions to the core 202 for execution. The cache memory 210 may provide data for processing by the core 202. In some examples, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to one or more processors included herein, e.g., processor 126. Controller 214 may control the data paths in the ALU 204, FPLU 206 and/or DSPU 208. Controller 214 may be implemented as one or more state machines, data paths and/or dedicated control logic. The gates of controller 214 may be implemented as standalone gates, FPGA, ASIC or any other suitable technology.

The registers 212 and the cache memory 210 may communicate with controller 214 and core 202 via internal connections 220A, 220B, 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache 210, and/or register 212. The bus 216 may be coupled to one or more components of the system.

The bus 216 may be coupled to one or more external memories. The external memories may include Read Only Memory (ROM) 232. ROM 232 may be a masked ROM, Electronically Programmable Read Only Memory (EPROM) or any other suitable technology. The external memory may include Random Access Memory (RAM) 1333. RAM 233 may be a static RAM, battery backed up static RAM, Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236.

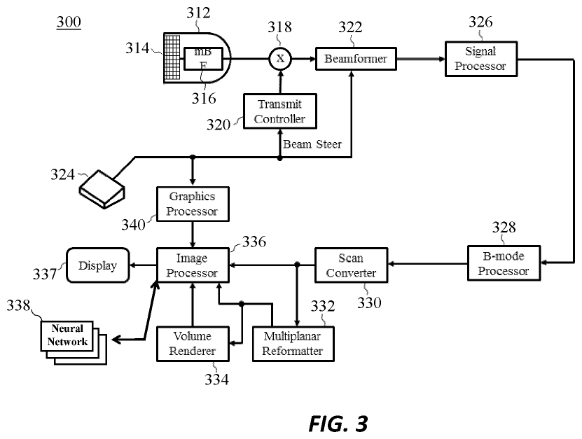

FIG. 3 is a block diagram of another ultrasound system 300 in accordance with principles of the present disclosure. One or more components shown in FIG. 3 may be included within a system configured to determine subject classes based on image acquisition settings and subsequently implement appropriate neural networks to process newly acquired images. For example, one or more of the above-described functions of data processor 126 and/or ultrasound data acquisition unit 110 may be implemented and/or controlled by one or more of the processing components shown in FIG. 3, including for example, signal processor 326, B-mode processor 328, scan converter 330, multiplanar reformatter 332, volume renderer 334 and/or image processor 336.

In the system of FIG. 3, an ultrasound probe 312 includes a transducer array 314 for transmitting ultrasonic waves into a targeted region of a subject pursuant to a scan protocol and receiving echo information responsive to the transmitted waves. In various embodiments, the transducer array 314 may be a matrix array or a one-dimensional linear array. The transducer array 314 may be coupled to a microbeamformer 316 in the probe 312, which may control the transmission and reception of signals by the transducer elements in the array. In the example shown, the microbeamformer 316 is coupled by the probe cable to a transmit/receive (T/R) switch 318, which switches between transmission and reception and protects the main beamformer 322 from high energy transmit signals. In some embodiments, the T/R switch 318 and other elements in the system can be included in the transducer probe rather than in a separate ultrasound system component. The transmission of ultrasonic beams from the transducer array 314 under control of the microbeamformer 316 may be directed by the transmit controller 320 coupled to the T/R switch 318 and the beamformer 322, which receives input, e.g., from the user's operation of the user interface or control panel 324. A function that may be controlled by the transmit controller 320 is the direction in which beams are steered. Beams may be steered straight ahead from (orthogonal to) the transducer array, or at different angles for a wider field of view. The partially beamformed signals produced by the microbeamformer 316 are coupled to a main beamformer 322 where partially beamformed signals from individual patches of transducer elements are combined into a fully beamformed signal.

The beamformed signals may be communicated to a signal processor 326. The signal processor 326 may process the received echo signals in various ways, such as bandpass filtering, decimation, I and Q component separation, and/or harmonic signal separation. The signal processor 326 may also perform additional signal enhancement via speckle reduction, signal compounding, and/or noise elimination. In some examples, data generated by the different processing techniques employed by the signal processor 326 may be used by a data processor and/or at least one neural network to identify one or more anatomical features, ultrasound artifacts, and/or image views. The processed signals may be coupled to a B-mode processor 328, which may employ amplitude detection for imaging structures in the body. The signals produced by the B-mode processor 328 may be coupled to a scan converter 330 and a multiplanar reformatter 332. The scan converter 330 may arrange the echo signals in the spatial relationship from which they were received in a desired image format. For instance, the scan converter 330 may arrange the echo signals into a two dimensional (2D) sector-shaped format. The multiplanar reformatter 332 may convert echoes which are received from points in a common plane in a volumetric region of the body into an ultrasonic image of that plane, as described in U.S. Pat. No. 6,443,896 (Detmer). In some examples, a volume renderer 334 may convert the echo signals of a 3D data set into a projected 3D image as viewed from a given reference point, e.g., as described in U.S. Pat. No. 6,530,885 (Entrekin et al.). The 2D or 3D images may be communicated from the scan converter 330, multiplanar reformatter 332, and volume renderer 334 to an image processor 336 for further enhancement, buffering and/or temporary storage for display on an image display 337.

Prior to their display, each of the images may be input and processed by one of a plurality of neural networks 338 trained to process each image from a particular subject class and generate one or more outputs based on the same. In embodiments, each of the plurality of neural networks 338 may be implemented at various processing stages, e.g., prior to the processing performed by the image processor 336, volume renderer 334, multiplanar reformatter 332, and/or scan converter 330.

A graphics processor 340 can generate graphic overlays for display with the processed ultrasound images. These graphic overlays may contain, e.g., standard identifying information such as patient name, date and time of the image, imaging parameters, and the like, and also various outputs generated by each of the neural networks 338, such as one or more indicators conveying the presence, absence and/or identity of one or more anatomical features embodied in a current image and/or whether various anatomical features have been observed and/or measured and/or which anatomical features have yet to be observed and/or measured in accordance with a stored worklist. Graphic overlays may also include visual instructions, e.g., text and/or symbols, for guiding a user of the system 300 through an ultrasound scan in a manner necessary to obtain images and/or measurements required for a particular assessment. In some examples, the graphics processor may receive input from the user interface 324, such as a typed patient name or confirmation that an instruction displayed or emitted from the interface has been acknowledged and/or implemented by the user of the system 300. The user interface 324 may also receive input regarding subject characteristics, the selection of particular imaging modalities and the operating parameters included in such modalities, input prompting adjustments to the settings and/or parameters used by the system 300, input requesting additional instructions or assistance for performing an ultrasound scan, and/or input requesting that one or more ultrasound images be saved and/or transmitted to a remote receiver. The user interface 324 may also be coupled to the multiplanar reformatter 332 for selection and control of a display of multiple multiplanar reformatted (MPR) images.

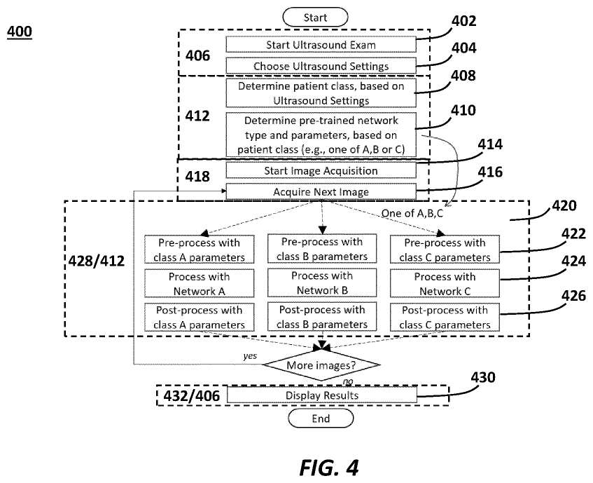

FIG. 4 depicts an example method 400 of ultrasound imaging, subject class determination, and automated network selection, as well as the ultrasound system components utilized to perform the method. As shown, the method 400 may involve initiating an ultrasound exam at step 402 and selecting ultrasound imaging settings at step 404. One or both of steps 402, 404 may be performed via a user interface 406 coupled with an ultrasound image acquisition unit. A patient or subject class (class A, B, or C) may then be determined at step 408 in view of the selected ultrasound settings, which may be utilized in combination with one or more subject characteristics. A pre-trained neural network (network A, B, or C) uniquely associated with the patient or subject class can then be determined at step 410, along with one or more parameters for processing the image frames acquired pursuant to the method 400. As further shown, steps 408 and/or 410 may be performed using at least one data processor 412.

Image acquisition begins at step 414 and the next image acquired at step 416 using an ultrasound image acquisition unit 418. Each acquired image is then assigned to a neural network corresponding to the subject class at step 420 and processed in accordance with a selected scan protocol. Processing each acquired image may involve several sub-processes. At step 422, for example, each image may be pre-processed with parameters unique to the subject class and associated neural network. Non-limiting, non-exhaustive pre-processing steps may involve normalizing scaling values and/or resizing image input for neural network compatibility. The pre-processed image is then processed with the assigned neural network at step 424 and post-processed using an additional set of parameters associated with the subject class at step 426. In some embodiments, post-processing may involve converting, formatting, and/or adjusting network-processed images and/or probabilities in accordance with user and/or system specifications. One or more of the processing steps included in step 420 may be performed by another data processor 428 or the same processor 412 used to determine subject class and associated neural network. If more images are needed or desired, the method returns to step 416 and is repeated therefrom. If additional image acquisition is not needed, the results may be displayed at step 430 on a user interface 432, which may comprise the same user interface 406 used to input the initial ultrasound settings. Neural network output may be displayed after processing each individual image frame or after results from a sufficient number of images have been accumulated for display, e.g., all frames from a short cine-loop clip. In some embodiments, the method 400 may further involve refining and eventually optimizing the thresholds applied for subject class determination, which may improve classification and processing of newly acquired images.

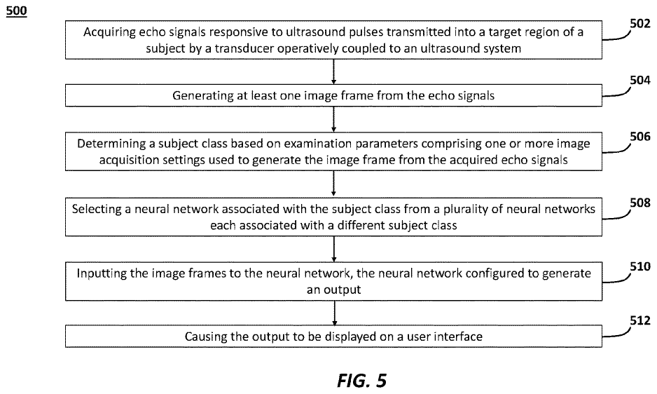

FIG. 5 is a flow diagram of a method of ultrasound imaging performed in accordance with principles of the present disclosure. The example method 500 shows the steps that may be utilized, in any sequence, by the systems and/or apparatuses described herein for determining a subject class and triggering implementation of a particular neural network trained to process acquired image frames corresponding to the subject class. The method 500 may be performed by an ultrasound imaging system, such as system 100, or other systems including, for example, a mobile system such as LUMIFY by Koninklijke Philips N.V. (“Philips”). Additional example systems may include SPARQ and/or EPIQ, also produced by Philips.

In the embodiment shown, the method begins at block 502 by “acquiring echo signals responsive to ultrasound pulses transmitted into a target region of a subject by a transducer operatively coupled to an ultrasound system.”

At block 504, the method involves “generating at least one image frame from the echo signals.”

At block 506, the method involves “determining a subject class based on examination parameters comprising one or more image acquisition settings used to generate the image frame from the acquired echo signals.”

At block 508, the method involves “selecting a neural network associated with the subject class from a plurality of neural networks each associated with a different subject class.”

At block 510, the method involves “inputting the image frames to the neural network, the neural network configured to generate an output.”

At block 512 the method involves “causing the output to be displayed on a user interface.”

Of course, it is to be appreciated that any one of the examples, embodiments or processes described herein may be combined with one or more other examples, embodiments and/or processes or be separated and/or performed amongst separate devices or device portions in accordance with the present systems, devices and methods.

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

Although the disclosed systems may have been described with particular reference to an ultrasound imaging system, it is also envisioned that the present system can be extended to other medical imaging systems where one or more images are obtained in a systematic manner. Accordingly, the present systems may be used to obtain and process images and associated information related but not limited to: testicular, breast, ovarian, uterine, thyroid, hepatic, lung, musculoskeletal, splenic, cardiac, arterial and vascular systems, as well as other imaging applications related to ultrasound-guided interventions. Further, the present systems may also include one or more programs which may be used with conventional imaging systems so that they may provide features and advantages of the present systems. Certain additional advantages and features of this disclosure may be apparent to those skilled in the art upon studying this disclosure, or may be experienced by persons employing the novel systems and methods of the present disclosure. Another advantage of the present systems and methods may be that conventional medical image systems can be easily upgraded to incorporate the features and advantages of the present systems, devices, and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound imaging system comprising:
an ultrasound transducer configured to acquire echo signals responsive to ultrasound pulses transmitted toward a subject; and
one or more processors in communication with the ultrasound transducer and configured to:
determine a subject class corresponding to the subject based on examination parameters comprising one or more image acquisition settings used to generate at least one image frame from the acquired echo signals;
select a neural network associated with the subject class from a plurality of neural networks each associated with a different subject class; and
input the at least one image frame to the neural network, the neural network configured to generate an output.

2. The system of claim 1, further comprising a user interface configured to receive indications of the examination parameters.

3. The system of claim 1, further comprising a controller coupled with the ultrasound transducer, the controller configured to implement the one or more image acquisition settings in the ultrasound transducer.

4. The system of claim 1, wherein the examination parameters further comprise one or more characteristics of the subject.

5. The system of claim 4, wherein the one or more characteristics of the subject comprise one or more of a body mass index (BMI), a weight, a developmental stage, an age, a height, or a health condition.

6. The system of claim 1, wherein the examination parameters further comprise an examination type comprising one or more of: a lung ultrasound, a cardiac ultrasound, an abdominal ultrasound, an obstetric ultrasound, a pelvic ultrasound, a transabdominal ultrasound, a transvaginal ultrasound, a transrectal ultrasound, a vascular ultrasound, a liver ultrasound, a renal ultrasound, a transcranial ultrasound, or a thyroid ultrasound.

7. The system of claim 1, wherein the one or more image acquisition settings comprise a gain, a scan depth, a probe type, a frequency, or a scan pattern.

8. The system of claim 2, wherein the user interface further comprises a display configured to display the output.

9. The system of claim 1, wherein the output comprises one or more of an ultrasound image, an identification of a natural or artificial feature visible within the ultrasound image, or a user instruction for performing an ultrasound exam.

10. The system of claim 1, wherein the one or more processors are configured to determine the subject class by applying one or more thresholds to the examination parameters.

11. The system of claim 10, wherein the one or more thresholds comprise a range of depth settings included in the image acquisition settings.

12. The method of claim 1, wherein each of the plurality of neural networks is operatively associated with a training algorithm configured to receive an array of training inputs and known outputs, wherein the training inputs comprise ultrasound image frames obtained from a distinct subject class.

13. A method of ultrasound imaging, the method comprising:
acquiring echo signals responsive to ultrasound pulses transmitted into a target region of a subject by a transducer operatively coupled to an ultrasound system;
generating at least one image frame from the echo signals;
determining a subject class based on examination parameters comprising one or more image acquisition settings used to generate the at least one image frame from the acquired echo signals;
selecting a neural network associated with the subject class from a plurality of neural networks each associated with a different subject class;
inputting the at least one image frame to the neural network, the neural network configured to generate an output; and
causing the output to be displayed on a user interface.

14. The method of claim 13, wherein the examination parameters further comprise one or more characteristics of the subject.

15. The method of claim 14, wherein the one or more characteristics of the subject comprise one or more of a body mass index (BMI), a weight, a developmental stage, an age, a height, or a health condition.

16. The method of claim 13, wherein the image acquisition settings comprise a gain, a scan depth, a probe type, a frequency, or a scan pattern.

17. The method of claim 13, wherein determining the subject class comprises applying one or more thresholds to the examination parameters.

18. The method of claim 17, wherein the one or more thresholds comprise a range of depth settings included in the image acquisition settings.

19. The method of claim 13, further comprising training each of the plurality of neural networks using an array of training inputs and known outputs, the training inputs comprising ultrasound image frames obtained from a distinct subject class.

20. A non-transitory computer-readable medium comprising executable instructions, which when executed cause one or more processors to perform any of the methods of claim 13.
